(19) United States
(12) Patent Application Publication
(10) Pub. No.: US 2002/0197686 A1
(43) Pub. Date: Dec. 26, 2002

(54) Water soluble carbhydrates derived from lignocellulose by enzyme hydrolysis

(76) Inventor: Gene E. Lightner, Federal Way, WA (US)

Correspondence Address: Gene E. Lightner, 706 S.W. 296th St., Federal Way, WA, 98023, US

(21) Appl. No.: 09/888,741

(22) Filed: Jun. 25, 2001

(51) Int. Cl. C12P 19/14; C12P 7/06

(52) U.S. Cl. 435/99; 435/161

(57) ABSTRACT

This is a method to create water soluble carbohydrates from lignocellulose containing cellulose by providing enzymes to the lignocellulose cellulose and subjecting the cellulose to hydrolysis. By combining an extractate, from a previous extraction, to the lignocellulose containing enzymes, hydrolysis of cellulose continues and creates water soluble carbohydrates and produces a residue containing lignins. Filtering the residue from water soluble carbohydrates containing enzymes will produce a filtrate and a filtered residue. The filtered residue is subjected to extraction by water to provide a water extracted residue and an extractate for recycle in subsequent hydrolysis, The filtrate containing water soluble carbohydrates and enzymes, is subjected to membrane filtration to substantially separate enzymes from the water soluble carbohydrates and produce enzymes for recycle and water soluble carbohydrates substantially devoid of enzymes. Thereby, water soluble carbohydrates are formed from lignocellulose and a residue containing lignins substantially devoid of water soluble carbohydrates is formed.

### BACKGROUND OF THE INVENTION

Biomass is associated with non-fossil organic materials that contain fundamental energy derived from the sun. Biomass, frequently termed lignocellulose, is often selected from the group consisting of wood, waste paper and municipal solid waste including an individual or a combination thereof. To avoid use of corrosive. acids it is desirable to apply cellulase for hydrolysis of cellulose contained in biomass to produce sugars for subsequent fermentation.

Biomass containing hemicellulose and cellulose does not have pores readily accessible by cellulose. Removal of hemicellulose from a biomass creates pores readily accessible by cellulase and a lignocellulose containing lignins and cellulose. A means of removal of hemicellulose from a biomass is described by Grothmann, et al, in U.S. Pat. No. 5,125,977 wherein two stages are employed relying on dilute acid to remove hemicellulose from a biomass and subject lignin to self-condensation to produce a pretreated porous biomass.

Hydrolysis of sterilized cellulose within lignocellulose, obtained from biomass frequently rely on cellulase enzymes for hydrolysis. This procedure is related by Wilke, et al, in U.S. Pat. No. 3,972,775 wherein cellulase contained in sugar solution, obtained by hydrolysis, is absorbed by cellulose contained within cellulosic materials. Cellulosic materials containing absorbed cellulase are subjected to “a continuous process for enzymatically converting a cellulosic material to soluble sugars”. Thus absorption of cellulase on cellulose is the means described for hydrolysis to create soluble sugars. Entitled “Simultaneous saccharification and fermentation (SSF) using cellibios fermenting yeast Brettanomyces custersii,” is a process presented within U.S. Pat. No. 5,100,791 by Spindler, et al. The process is accomplished by a temperature less than allowed for rate of hydrolysis by cellulases. Accordingly the process does not establish temperature conditions for highest rate of hydrolysis. It is therefore an object of this invention to obviate the limitations or disadvantages of the prior art.

A distinct object of this invention is to employ cellulase for hydrolysis of cellulose contained within lignocellulose to create water soluble carbohydrates

A further object of this invention is to employ membranes to separate cellulases from mixtures contained within water soluble carbohydrates

Another object of this invention is to establish and maintain temperature and pH conditions for highest rate of hydrolysis allowed by cellulase for hydrolysis.

With the above and other objects in view, this invention relates to the novel features and alternatives and combinations presently described in the brief description of the invention.

### APPLICATIONS OF THE INVENTION

Principles applied to present invention to produce water soluble carbohydrates derived from a biomass include;

Hemicellulose can be substantially removed from biomass containing hemicellulose by hydrolysis Pretreatment of biomass by dilute acid, described by Grothmann et al, page B-15 in Biochemical Conversion/Alcohol Fuels Program Annual Report, November, 1988, removes hemicellulose concurrent to condensation of lignin and creates a lignocellulose with a porous consistency and renders cellulose readily accessible to cellulase enzymes, or equivalent enzymes. Accordingly hemicellulose, forming a barrier for cellulase enzymes, is removed.

Hydrolysis of cellulose by cellulases is optimized at a pH of about 4-5 and a temperature of 40-50 degrees Celsius required for hydrolysis to form water soluble glucose polymers reported in Cellulase on the internet.

Separation of water soluble glucose polymers, containing cellulase enzymes, depend on a membrane to separate, by a difference in molecular weight, to produce a filtrate of water soluble glucose polymers substantially devoid of enzymes and a concentrate of high molecular weight enzymes. The operation for this membrane separation is termed ultafiltration as described in Ultafiltration Application Bulletin 112 Organics Jun. 4, 1996. The concentrate of enzymes accordingly separated from a mixture of water soluble glucose polymers and enzymes is ready to be recycled.

Filtrate of water soluble glucose polymers, substantially devoid of enzymes, is thus created.

### BRIEF DESCRIPTION OF THE INVENTION

The present invention in its broadest aspect, establishes a method to produce water soluble carbohydrates by hydrolysis of cellulose contained within a lignocellulose. Hydrolysis of cellulose contained within a lignocellulose is accomplished by enzymes to create a mixture of water soluble carbohydrates and enzymes. Upon hydrolysis the residue containing lignins is filtered to produce a filtrate and a residue for extraction with water. The filtered residue is extracted by water to produce an extractate and a water extracted residue. The extractate is recycled, and combined with enzymes and maintained at a pH of about 5 and a temperature of about 40-50 degrees Celsius. Consequent to providing a membrane to divide the filtrate of water soluble carbohydrates and enzymes to produce water soluble carbohydrates substantially devoid of enzymes and provides a concentrate of enzymes for recycle. The filtrate containing water soluble carbohydrates and enzymes is frequently subjected to absorption by cellulose within lignocellulose to provide absorbed enzymes for hydrolysis of cellulose contained in a lignocellulose and a diluted filtrate, containing a diminished content of enzymes, subjected to separation by the provided membrane to produce water soluble carbohydrates substantially devoid of enzymes and enzymes separated from the membrane for recycle. The enzymes are often selected from the group consisting of cellulase, glucanhydrolase and, cellobiohydrolase including an individual or a combination thereof Hydrolysis is frequently accomplished in a vessel to constitute a continuous method.

Characteristics of the invention include:

Hydrolysis of cellulose contained within a lignocellulose is promoted by enzymes to create a mixture of water soluble carbohydrates and enzymes.

Residue, containing lignins from hydrolysis, is filtered to produce a filtered residue and a filtrate.

The filtered residue is extracted by water to form an extractate and a water extracted residue.

The mixture of water soluble carbohydrates and enzymes, subjected to molecular weight separation by a membrane provides enzymes for recycle and water soluble carbohydrates substantially devoid of enzymes.

Temperature and pH are sustained within hydrolysis to maintain maximum rate of hydrolysis.

### BRIEF DESCRIPTION OF THE DRAWINGS

The features that are considered characteristic of this invention are set forth in the appended claims. This invention, however, both as to its origination and method of operations as well as additional advantages will best be understood from the following description when read in conjunction with the accompanying drawings in which:

FIG. 1 is a flow sheet denoting the invention as set forth in the appended claims.

FIG. 2 is a flow sheet denoting a method to produce a porous lignocellulose from a biomass.

FIG. 3 is a flow sheet denoting a method to produce glucose and ethanol from water soluble carbohydrates.

### DETAILED DESCRIPTION OF THE PREFERRED EMBODIMENT

In the preferred embodiment of the present invention, a means of producing water soluble carbohydrates derived from lignocellulose is presented. The water soluble carbohydrates are derived by hydrolysis of cellulose contained in a lignocellulose by enzymes.

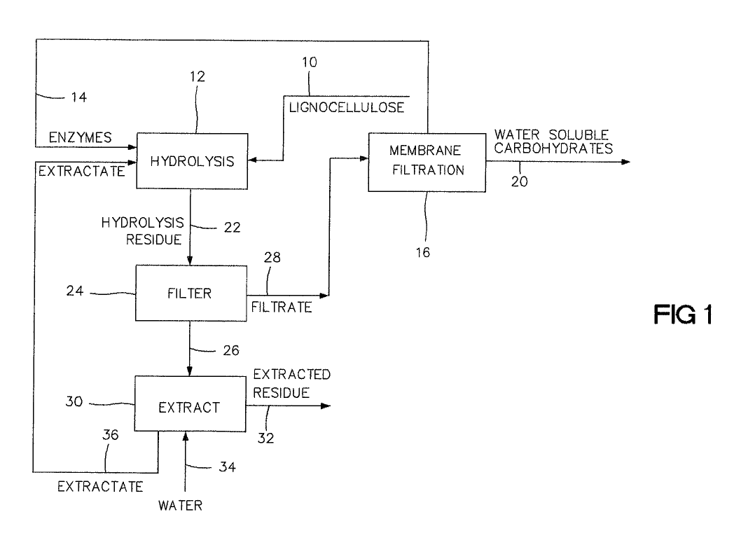

The flow diagram of FIG. 1 illustrates the general preferred embodiment of the present invention. In the diagram, rectangles represent stages, operations or functions of the present invention and not necessarily separate components. Arrows indicate direction of flow of material within the method.

Referring to FIG. 1, lignocellulose 10 is conveyed to hydrolysis stage 12 and combined with enzymes 14 and subjected to hydrolysis. Hydrolysis residue 22 from hydrolysis stage 12 is conveyed to filter stage 24 and filtered to produce filtrate 28 and filtered residue 26 conveyed to extract stage 30 Residue 26 is extracted by water 34 to produce extractate 36 and extracted residue 32. Extractate 36 is conveyed to hydrolysis stage 12. Filtrate 28 is conveyed to membrane filtration stage 16 which is employed to separate dissimilar molecular weights to produce a solution of water soluble carbohydrates substantially devoid of enzymes 20 and produce enzymes 14 to be conveyed to hydrolysis stage for recycle 12. Membrane filtration stage 16 is generally an ultrafiltration membrane employed to separate dissimilar molecular weights. Hydrolysis of cellulose contained in lignocellulose is achieved by cellulase type enzymes to yield water soluble carbohydrates. Temperature and pH are adjusted within the hydrolysis environment to accomplish maximum hydrolysis rate allowed. Porous lignocellulose, containing cellulose, renders cellulose readily accessible to cellulase enzymes.

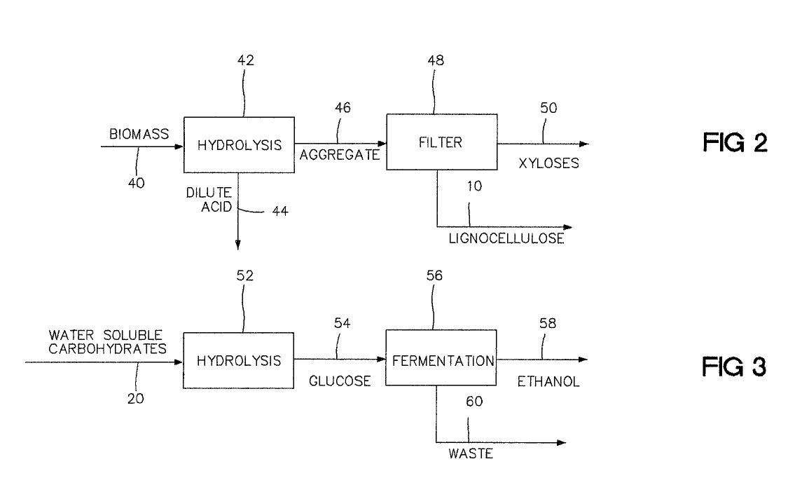

Referring to FIG. 2, biomass 40 is transported to hydrolysis stage 42, combined with dilute acid 44 to attain hydrolysis of hemicellulose, contained within biomass, 40. Aggregate 48, formed by hydrolysis, is transported to filter stage 48. Filter stage 48 performs filtration to form lignocellulose 10 and xyloses 50. Hemicellulose, contained in biomass, is accordingly hydrolyzed to produce lignocellulose 10 substantially devoid of hemicellulose. Lignocellulose 10, substantially lacking in hemicellulose, is accordingly rendered porous to improve absorption of enzymes by cellulose contained within lignocellulose.

Acid within lignocellulose 10 and xyloses 50 is substantially neutralized. Xyloses 50, when neutralized, are subjected to fermentation to form ethanol. Hydrolysis stage 44 may be configured as two stages to recognize relative ease and relative difficulty of hemicellulose hydrolysis to prevent or limit formation of furfural.

Referring to FIG. 3, water soluble carbohydrates 20 are transported to hydrolysis stage 52 to achieve hydrolysis and produce glucose 54, then transported to fermentation stage 56, to accomplish fermentation to produce ethanol 58 and produce waste 60. Hydrolysis of water soluble carbohydrates 20 may be achieved by enzymes or within an acidic environment. Waste 60, ultimately discarded, includes carbon dioxide produced by fermentation. Waste 60, is separated from ethanol contained within the waste before disposal of the waste.

What is claimed is:

1. A method to produce water soluble carbohydrates from lignocellulose,
which comprises:
providing lignocellulose containing cellulose and
providing enzymes to said cellulose, and
providing a membrane to divide a filtrate, and
combining the extractate, from a previous extraction, with said lignocellulose, and
subjecting said cellulose, contained in lignocellulose, to hydrolysis, at a pH of about 5, by said enzymes to create said water soluble carbohydrates and produce a residue containing lignins, and
filtering said residue containing lignins from said water soluble carbohydrates containing enzymes to produce a filtrate and a filtered residue, and
extracting the filtered residue containing lignins with water to substantially extract water soluble carbohydrates from the residue to produce a water extracted residue and an extractate for recycle, and
employing said membrane to substantially divide said filtrate containing water soluble carbohydrates and enzymes to provide water soluble carbohydrates substantially devoid of enzymes and provide enzymes for hydrolysis of cellulose contained in said lignocellulose thereby water soluble carbohydrates substantially devoid of enzymes are formed from lignocellulose and a residue containing lignins substantially devoid of water soluble carbohydrates is formed.

2. The method of claim 1 wherein said hydrolysis is accomplished in a vessel.

3. The method of claim 1 wherein said filtrate is subjected to ultrafiltration to substantially separate enzymes from the water soluble carbohydrates and form a solution substantially devoid of enzymes and recycle the separated enzymes for subsequent hydrolysis of cellulose contained in a lignocellulose.

4. The method of claim 3 wherein the, solution containing water soluble carbohydrates, is subjected to hydrolysis and fermentation to form ethanol.

5. The method of claim 1 wherein said lignocellulose is obtained from biomass selected from the group consisting of wood, waste paper and municipal solid waste including an individual or a combination thereof.

6 The method of claim 1 wherein said lignocellulose is provided from dilute acid hydrolysis of a biomass to provide a lignocellulose substantially devoid of hemicellulose.

7 The method of claim 1 wherein said enzymes are selected from the group consisting of cellulase, glucanhydrolase and, cellobiohydrolase including an individual or a combination thereof.

8. The method of claim 1 wherein said lignocellulose containing cellulose is accessible to enzymes.

9. The method of claim 1 wherein said extractate contains water soluble carbohydrates.

10. The method of claim 1 wherein said water soluble carbohydrates contain glucose.

11. The method of claim 1 wherein said water soluble carbohydrates contain glucose polymers.

12. The method of claim 1 wherein said water soluble carbohydrates contain cellodextrins.

13. The method of claim 1 wherein said enzymes derived from ultrafiltration are recycled to provide enzymes to said cellulose contained in a lignocellulose.

14. The method of claim 1 wherein said water soluble carbohydrates containing enzymes are absorbed by cellulose to provide absorbed enzymes for hydrolysis of cellulose contained in a lignocellulose.

15. The method of claim 1 wherein said water soluble carbohydrates are subjected to hydrolysis to form glucose.

16. The method of claim 1 wherein said method is continuous.

17. The method of claim 1 wherein said lignocellulose is obtained from pretreated biomass.

18. The method of claim 1 wherein said lignocellulose is substantially devoid of hemicellulose.

19. The method of claim 1 wherein said lignocellulose is substantially sterilized.
